(19) United States
(12) Patent Application Publication
(10) Pub. No.: US 2013/0063699 A1
(43) Pub. Date: Mar. 14, 2013

(54) Ocular Error Detection

(71) Applicants: Ervin Goldfain, Syracuse, NY (US); Corinn C. Fahrenkrug, Chittenango, NY (US); Daniel C. Briggs, Memphis, NY (US)

(72) Inventors: Ervin Goldfain, Syracuse, NY (US); Corinn C. Fahrenkrug, Chittenango, NY (US); Daniel C. Briggs, Memphis, NY (US)

(73) Assignee: WELCH ALLYN, INC., Skaneateles Falls, NY (US)

(21) Appl. No.: 13/487,959

(22) Filed: Jun. 4, 2012

Related U.S. Application Data
(60) Provisional application No. 61/532,702, filed on Sep. 9, 2011.

(51) Int. Cl. A61B 3/103

(52) U.S. Cl. 351/221; 351/246

(57) ABSTRACT

A system for determining refractive eye aberrations includes an optical arrangement having a first conjugate lens having an effective focal length (EFL) of about 150 millimeters and a second conjugate lens having an EFL of about 88.9 millimeters. The first and second conjugate lens are each positioned in a housing along a return light path and are separated by a distance of about 238.9 millimeters. The arrangement enables a range of measurable diopters of an eye to be between about −10 diopters to about +10 diopters.

### CROSS REFERENCE TO RELATED APPLICATION

This application claims the benefit of U.S. patent application Ser. No. 61/532,702 entitled “Ocular Error Detection,” filed 9 Sep. 2011, the entirety of which is hereby incorporated by reference.

This application is related to U.S. patent application Ser. No. 09/089,807 entitled “Compact Ocular Measuring System,” filed 3 Jun. 1998, the entirety of which is hereby incorporated by reference.

### BACKGROUND

Ocular measuring systems provide an easy and convenient way for healthcare professionals to screen for vision problems such as, for example, near and farsightedness (myopia/hyperopia), astigmatism (asymmetrical focus), and anisometropia (unequal power between eyes). The ease of use of such systems also makes them ideal for screening infants or handicapped patients in either a medical office or otherwise offsite setting.

### SUMMARY

In one aspect, a first apparatus for determining refractive eye aberrations includes: a housing; an illumination source positioned in the housing and configured to project a beam of light into an eye of a patient along an illumination axis, the beam forming a secondary source on a back portion of the eye for a return light path of an outgoing wavefront from the eye; a sensor positioned in the housing and along the return light path, the sensor including a light detection surface; a first lens and a second lens each positioned in the housing along the return light path, where the first lens includes a first focal length of about 150 millimeters and the second lens includes a second focal length of about 88.9 millimeters, and where the first and second lens are separated by a distance of about 238.9 millimeters; an optics array positioned between the sensor and the first and second lens in the housing along the return light path, where the optics array includes a plurality of lenslets positioned to focus portions of the wavefront onto the light detection surface, and where the sensor is configured to detect deviations in positions of the focused portions impinging the light detection surface to determine aberrations of the wavefront; and a viewer positioned in the housing and configured to align the eye with the illumination axis.

In another aspect, a method of measuring refractive eye error includes: projecting a beam of light into an eye, the light producing a secondary source and generating a wavefront from the eye along a return light path; directing the wavefront through a first lens and a second lens onto an optics array having a series of planarly positioned lenslet elements, where the first lens includes a first focal length of about 150 millimeters and the second lens includes a second focal length of about 88.9 millimeters, and where the first and second lens are separated by a distance of about 238.9 millimeters; focusing incremental portions of the generated wavefront passing through the lenslet elements onto an imaging substrate; and measuring deviations in the incremental portions of the generated wavefront on the imaging substrate to measure refractive error in the eye.

In yet another aspect, a second apparatus for determining refractive eye aberrations includes: a housing; a laser diode positioned in the housing and configured to emit a light beam into an eye of a patient along an illumination axis, the light beam having a wavelength in a range of about 750 nanometers to about 850 nanometers and forming a secondary source on a back portion of the eye for a return light path of an outgoing wavefront from the eye; a sensor positioned in the housing and along the return light path, the sensor including a light detection surface; a first lens and a second lens each positioned in the housing along the return light path, where the first lens includes a first focal length of about 150 millimeters and the second lens includes a second focal length of about 88.9 millimeters, and where the first and second lens are separated by a distance of about 238.9 millimeters; an optics array positioned between the sensor and the first and second lens in the housing along the return light path, wherein the optics array includes a plurality of lenslets positioned to focus portions of the wavefront onto the light detection surface, and where the sensor is configured to detect deviations in positions of the focused portions impinging the light detection surface to determine aberrations of the wavefront; an ultrasonic sensor positioned on the housing, the ultrasonic sensor configured to produce at least one audible signal based on a distance between the housing and the eye; a viewer positioned in the housing and configured to align the eye with the illumination axis, where the viewer is further positioned along a viewing axis, the viewing axis arranged at an oblique angle relative to the illumination axis; and a display configured to display data measured by the light detecting surface. A range of measurable diopters of the eye is about −10 diopters to about +10 diopters.

This Summary is provided to introduce a selection of concepts, in a simplified form, that are further described below in the Detailed Description. This Summary is not intended to be used in any way to limit the scope of the claimed subject matter. Rather, the claimed subject matter is defined by the language set forth in the Claims of the present disclosure.

### DESCRIPTION OF THE DRAWINGS

FIG. 1 is a schematic view illustrating differences between generated wavefronts exiting from an ideal eye and an aberrated eye, respectively.

FIG. 2 is a diagrammatic view of a refractive error measuring system in accordance with one embodiment of the present disclosure.

FIG. 3 is a partial schematic view of the microoptics array of the system of FIG. 2.

FIG. 4 is a block diagram representative of the refractive error measuring system of FIG. 2.

FIG. 5 is a ray trace diagram of the illumination portion of the system illustrated in FIG. 4.

FIG. 6 is a ray trace diagram of the measurement portion of the system illustrated in FIG. 4.

FIG. 7 is a ray trace diagram of the unfolded viewing portion of the system illustrated in FIG. 4.

FIG. 8 is a partial interior view of the refractive error measuring system according to one embodiment according to the present disclosure.

FIG. 9 is a partial interior view of another embodiment of a refractive error measuring apparatus according to the present disclosure.

FIG. 10 is a partial side view of the refractive error measuring system of FIG. 8.

FIG. 11 is a partial ray trace diagram of the folded viewing portion of the system of FIG. 8.

FIG. 12 is an example system for calibrating a refractive error measuring system.

FIG. 13 is another view of the system of FIG. 12.

FIG. 14 is another example system for calibrating a refractive error measuring system.

FIG. 15 is another view of the system of FIG. 14.

### DETAILED DESCRIPTION

The present disclosure is generally directed to systems and methods for determining refractive eye aberrations. In one example embodiment, an optical arrangement including a first conjugate lens having an effective focal length (EFL) of about 150 millimeters and a second conjugate lens having an EFL of about 88.9 millimeters are each positioned in a housing along a return light path. The first and second conjugate lenses are separated by a distance of about 238.9 millimeters. The example arrangement beneficially enables a range of measurable diopters of an eye to be between about −10 diopters to about +10 diopters. Although not so limited, an appreciation of the various aspects of the present disclosure will be gained through a discussion of the examples provided below.

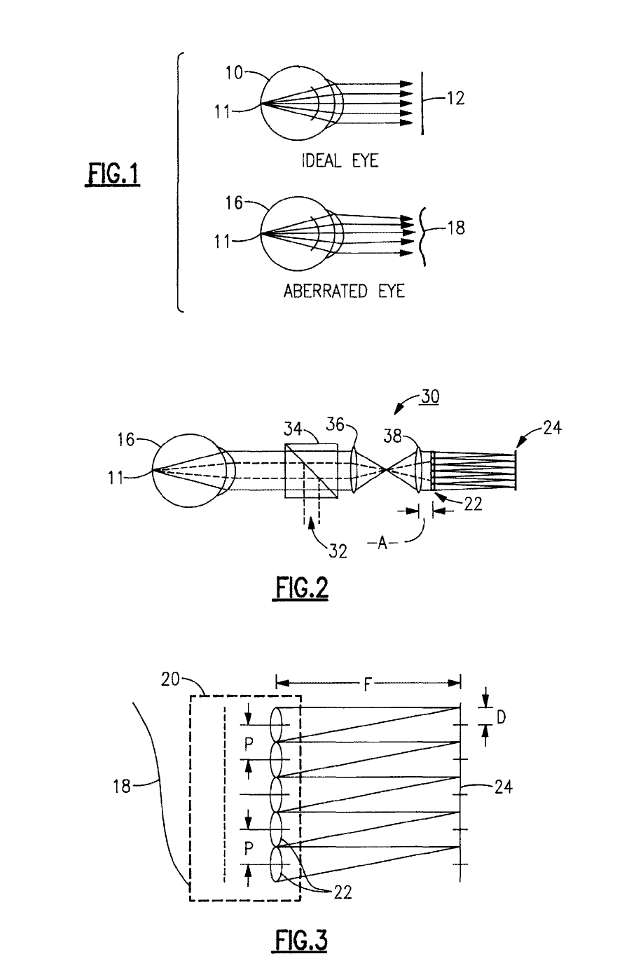

For purposes of background, reference is first made to FIG. 1. When a beam of light is projected into an eye of interest, the light is focused onto the back of the eye by optics thereof and diffusely reflected by the retina. The outgoing beam is more or less focused and forms a secondary source 11 for light which exits the eye and generates a wavefront, as shown in FIG. 1. Herein, a secondary source is referred to as the image of the illuminating source or the fiducial mark (if used) onto the back of the eye created by the illuminating optics. The wavefront 12 of an ideal eye 10; that is, an eye substantially free from refractive errors, is defined by a set of substantially outgoing collimated rays and thereby forms a planar wavefront. On the other hand, the wavefront 18 generated by an aberrated eye 16 is defined by a series of non-collimated outgoing rays, generating a wavefront which deviates from the ideal planar form.

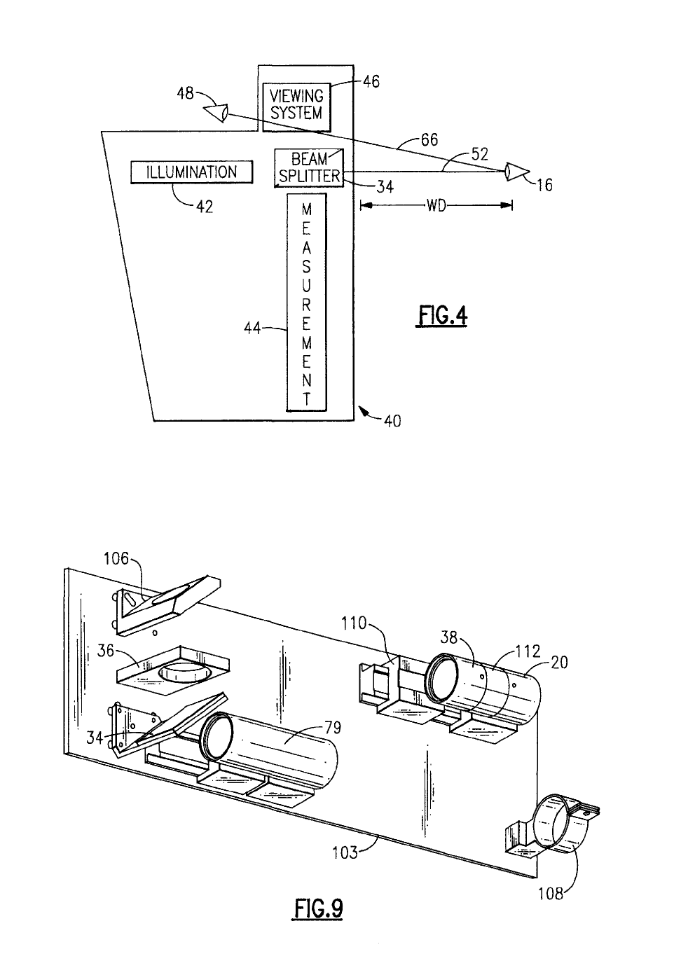

Referring to FIG. 2, a diagrammatic view is presented of a refractive error measuring system 30 in accordance with the present application. A more detailed description follows, but in brief, a substantially collimated beam of light 32 is passed through a beam splitter 34 along an illumination axis which is then directed to the eye of interest. The collimated beam of light 32 is focused as a secondary source 11 on the back of the eye 16, thereby producing the generated wavefront 18, FIG. 1, exiting from the eye along a return light path. The beam of light 32 according to a preferred arrangement can be adjusted, e.g. converged/diverged to adjust the point of focus, such as for young children.

A pair of conjugate lenses, 36, 38, described in greater detail below, direct the light to a microoptics array 20 where each of the incremental portions of the generated wavefront 18 are substantially focused onto an imaging substrate 24.

FIG. 3 shows microoptics array 20 containing a plurality of small planarly disposed lenslets 22. Each of the lenslets 22 is evenly separated from one another by a dimension -P-, hereinafter referred to as pitch. Light from the generated wavefront 18, FIG. 1, entering the microoptics array 20 is focused by the lenslets 22 onto an imaging substrate 24 or other detecting surface which is preferably placed at a suitable distance -F- from the lenslet elements. Incremental portions of the wavefront 18, FIG. 1, passing through a sufficient number of lenslets 22 are then focused onto the imaging substrate 24 and the deviations -D- of the positions of the incremental portions relative to a known zero or “true” position can be used to compute refractive error relative to a known zero or ideal diopter value. This can be defined as an array of “zero” spots corresponding to a planar wavefront, such as that shown in FIG. 1. Details relating to an example mathematical technique for estimating the wavefront are described in detail within W. H. Southwell, “Wave-front estimation from wave-front slope measurements,” J. Opt. Soc. Am. 70, 998-1006 (1980), and (b) Junzhong Liang, Bernhard Grimm, Stefan Goelz, and Josef F. Bille, “Objective measurement of wave aberrations of the human eye with the use of a Hartmann-Shack wave-front sensor,” J. Opt. Soc. Am. A 11, 1949-1957 (1994), the entireties of which are hereby incorporated by reference.

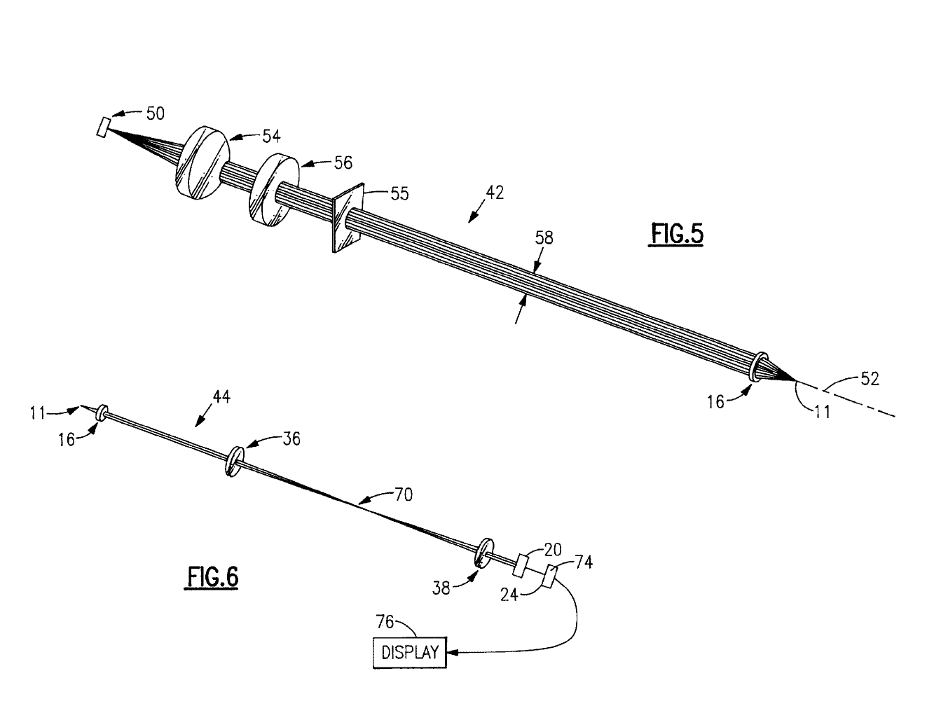

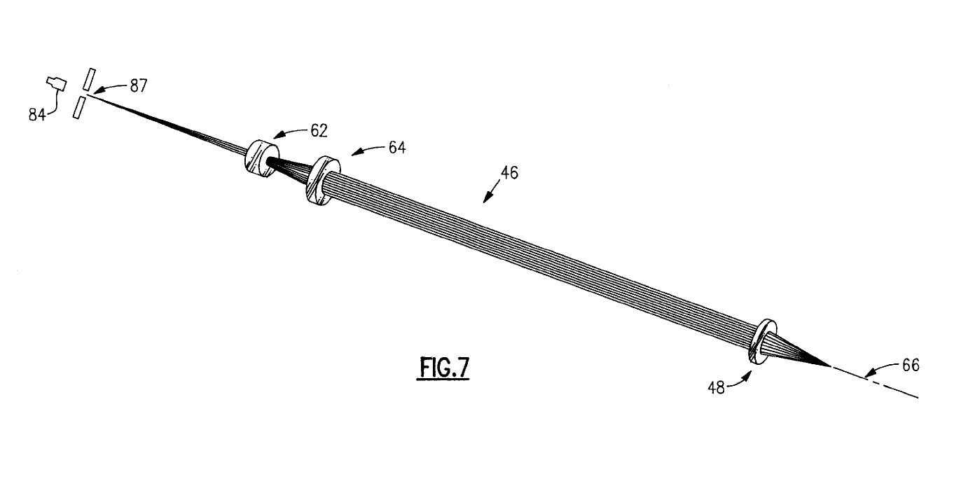

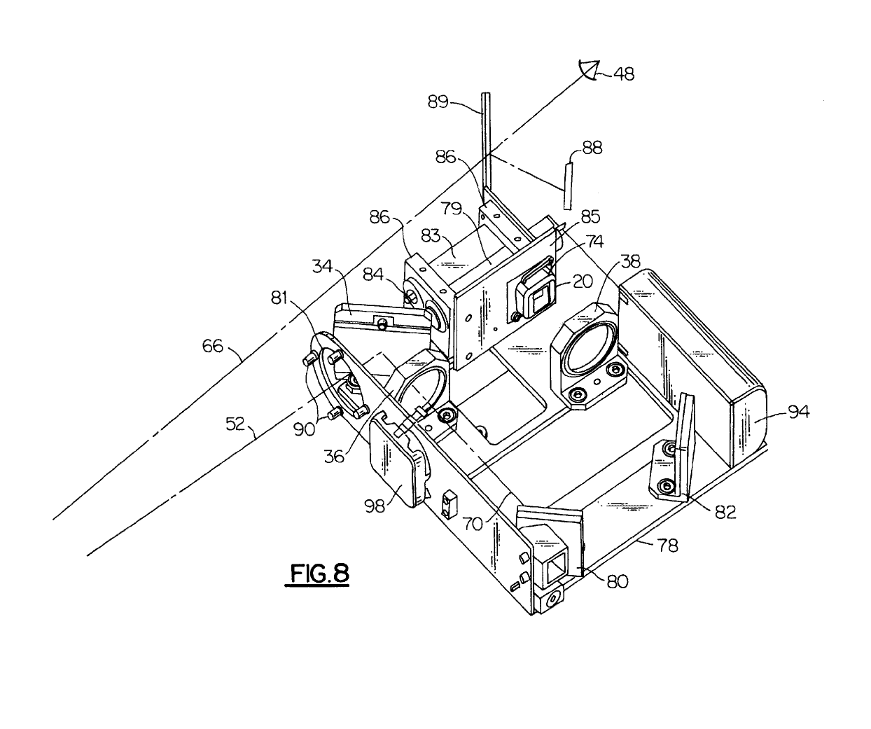

A block diagram of the apparatus according to the present application is herein described with reference to FIG. 4 including a housing 40 having an interior sized for containing the described system 30, FIG. 2, having in particular three major subassemblies; namely an illumination subassembly 42, a measurement subassembly 44, and a viewing subassembly 46 shown relative to a viewing eye 48. One embodiment of each subassembly is shown in the following FIGS. 5-7 for use in the instrument housings, shown more particularly in FIGS. 8 and 9. An important feature of the present application is that the instrument housing 40 can be situated for operation at a suitable working distance -WD- from the eye 16 of the patient. According to one embodiment, a working distance of approximately 40 cm is suitable.

Each of the subassemblies 42, 44, 46 will be described prior to describing structural embodiments which employ the described subassemblies. Referring first to FIG. 5, a schematic diagram is shown for the illumination assembly 42, the purpose of which is to focus a beam of light onto the back of the eye 16; that is, onto the retina of a patient. According to this embodiment, a laser diode 50 is preferably used as an illumination source which projects monochromatic light in conjunction with a plano-convex singlet 54 disposed adjacent to a plano-concave singlet 56, the elements being arranged and aligned to produce a beam of substantially collimated light 58 which can be projected along the illumination axis 52 into the eye of interest, the light being focused onto the back thereof, as previously shown in FIG. 1.

More specifically, and according to this embodiment, the plano-convex singlet 54 and the plano-concave singlet 56 have effective focal lengths of approximately 25 mm and −50 mm, respectively, closed with an aperture 55 to produce a substantially collimated beam of light having a diameter of approximately 2.5 mm. The laser diode 50 emits near-infrared light having a wavelength of approximately 780 nm, so as not to constrict the pupil. Alternately, a halogen (or other broad-band) illumination source (not shown) could be substituted with adequate filtering. Still other lens systems could be utilized in lieu of the one herein described; for example, a single lens having a 60 mm effective focal length could be substituted for the lens pair of the embodiment.

By modifying the distances between the plano-convex singlet 54 and the plano-concave singlet 56, the beam of light projected can be made to be slightly divergent, or slightly convergent. This variation will create a best focus on the back of an eye which is slightly myopic or hyperopic, respectively. Illumination adjustment allows the system to be optimized for a likely refractive range of a targeted population.

As shown in FIG. 7, a schematic diagram of the major optical components of the viewing subassembly 46 is shown which is used to align the viewing eye 48 to the illumination axis 52, FIG. 5, of the illuminating assembly 42, FIG. 5. The optics of the viewing subassembly 46 of the example embodiment include a plano-concave singlet 62 which is disposed adjacent to a plano-convex singlet 64.

According to one embodiment shown, the first singlet 62 has an effective focal length of −8 mm, while the second singlet 64 has an effective focal length of approximately 22 mm. It should be apparent, however, that these parameters can also easily be varied.

As shown more clearly in the structural version of the apparatus shown in FIG. 8, the viewing subassembly 46, shown in phantom, is maintained either at a side or at a height above the collimated light 58, of FIG. 5 (approximately 8 degrees according to this embodiment).

As described more completely below, an alignment guide or pattern, such as a crosshairs (not shown), is targeted using a viewing window 89 which is aligned with a viewing port (not shown) and along a viewing axis 66 which is inclined relative to the illumination axis 52. Alternately, the viewing subassembly 46 can include an eyepiece (not shown) and magnifying optics (not shown).

Referring now to FIG. 6, the measurement subassembly 44 includes a number of components used to direct the generated wavefront 18, FIG. 1, along a return light path 70 from the eye 16. A pair of fixed conjugate lenses 36, 38 is placed between the eye of interest and the microoptics array 20 along the return light path 70. For purposes which are described in greater detail below, the conjugate pair is preferably separated from one another by substantially the sum of their respective and preferably unequal focal lengths.

According to one embodiment, the first conjugate lens 36 is a plano-convex element having a focal length of approximately 150 mm and the second conjugate lens 38, also a plano-convex element has a focal length of about 63 mm, providing a total distance therebetween of approximately 213 mm. In another embodiment, the first conjugate lens 36 is a plano-convex element having a focal length of about 150 millimeters and the second conjugate lens 38, also a plano-convex element, has a focal length of about 88.9 millimeters, providing a total distance therebetween of about 238.9 millimeters. In this example embodiment, the first conjugate lens 36 is an Edmund Optics NT32-864 lens and the second conjugate lens 38 is a JML Optical Industries CBX10659 lens. Other embodiments are possible.

The microoptics array 20 is further disposed along the return light path 70 from the second conjugate lens 38 and at a distance of approximately 17 mm from the second conjugate lens 38. An electronic sensor 74, such as a charge coupled device (CCD) or other imaging sensor having an imaging substrate 24 is then disposed at a predetermined distance therefrom.

According to one embodiment, the electronic sensor 74 is a Sony ICXO84AL, though other electronic imaging sensors, such as a Panasonic GP-MS-112 black and white video camera having either CCD or CMOS architecture, for example or others, can be substituted, each having appropriate processing circuitry as is known in the field, requiring no further discussion.

Referring to FIGS. 3 and 6, the microoptics array 20 according to one embodiment, such as those manufactured and sold by Adaptive Optics Inc, of Boston, Mass., comprises a matrix of lenslets 22 disposed in a planar relationship which when positioned in the return light path 70 is orthogonal thereto. According to one embodiment, the lenslets 22 each have an effective focal length of approximately 8 mm and are each separated from one another by approximately 0.50 mm. It will be readily apparent that each of these parameters can be suitably varied, for example, pitch in the range of approximately 0.25 mm to approximately 2 mm is adequate.

As previously noted, the incremental portions of the generated wavefront 18, FIG. 1, are substantially focused onto an imaging substrate 24 of the electronic sensor 74, which is disposed orthogonally to the return light path 70 and placed the predetermined distance -F- from the lenslets 22 of the microoptics array 20. Preferably, and according to this embodiment, the distance -F- between the microoptics array 20 and the imaging substrate 24 of the electronic sensor 74 is approximately 8 mm, which is the focal length of the lenslets 22.

In brief, light impinging on the imaging substrate 24 is detected by the electronic sensor 74 in a manner conventionally known. The image which is formed at the electronics sensor 74 consists of a matrix of spots, one for each lenslet of the lenslets 22. These spots are captured by the imaging substrate 24 at the distance -F- from the microoptics array 20. The distance -D-, FIG. 3, between the centroids of each of the spots is calculated and is used to determine the refractive power of the wavefront 18, FIG. 3, which created them. This power is corrected by the conjugate lens mapping function to interpolate the power at the eye. The optical power detected at the lenslet does not equal the optical power of the measured eye. Therefore, one needs to convert the diopter readings from the microoptics array to the patient's eye. This refractive error is reported to the user of the instrument through an attached LCD 76, shown schematically in FIG. 6. The principles for estimation of the formed wavefront, using Zernike polynomials are described in Journal of Optical Society of America, vol. 69, No. 7 in an article by Cubalchini, the entire contents of which are herein incorporated by reference.

Referring now to FIG. 8, a particular embodiment of the above apparatus is herein described employing the above subassemblies 42, FIG. 5, 44, FIG. 6. 46, FIG. 7. The apparatus is shown in part as mounted to a support plate 78 contained within the housing 40, FIG. 4, shown only partially for the sake of clarity in describing the embodiment. The basic components previously described in FIGS. 5-7 are utilized herein, but the return light path 70 is folded to maximize packaging into a conveniently sized housing.

The support plate 78 maintains each of the components herein described in a fixed relative position. The laser diode 50, FIG. 5, is supported within an illumination housing 79 along with suitable illuminating optics, such as described above with respect to FIG. 5, the illumination output being transmitted through a beam splitter 34 so as to project a beam of substantially collimated light along an illumination axis 52.

An adjacent housing 83 includes an LED 84 and aperture 87 for backlighting a cross-hair or other conveniently shaped alignment pattern (not shown), the pattern being placed in the viewing system and projected using a folding mirror 88 and a viewing window 89 disposed along the viewing axis 66 and aligned with the viewing eye 48.

The viewing subassembly 46 is intended to provide to the practitioner a means to align the device to the patient's pupil. The alignment pattern (not shown) is projected onto the viewing window 89 through a side train of lenses (not shown) and the folding mirror 88 such that the pattern appears to be at the same working distance as the patient's eye. According to this embodiment, the working distance -WD- is approximately 40 cm.

The entire viewing subassembly 46 is positioned off axis with respect to the illumination axis 52. The oblique position of the viewing subassembly 46 relative to the illumination subassembly 42 separates the viewing and illumination measurement paths, as opposed to a coaxial design which would require two or more beam splitters. Because of the relatively long working distance, the oblique position does not substantially affect the ability to align the patient's pupil to the optical axis of the instrument.

According to this embodiment, the main beam splitter 34 is disposed relative to the laser diode 50, FIG. 5, so as to be positioned 45 degrees relative to the illumination/measurement axis to direct light received from the eye of interest orthogonally along the return light path 70 to the first conjugate lens 36 mounted in a conventional manner to the support plate 78 and aligned with a pair of folding mirrors 80, 82 also aligned to fold the return light path, allowing convenient and compact packaging. The second conjugate lens 38 is disposed between the second folding mirror 82 and the microoptics array 20 which is attached along with the electronic sensor 74 to a vertical plate 85 attached to the support 86 for the illumination assembly and the LED generator housing 83 for the viewing assembly 46.

The return light path 70 therefore exits the eye 16, FIG. 2, and reenters the device through an existing port 81. The light is deflected by the beam splitter 34 and then is directed through the first conjugate lens 36 and is folded by the mirrors 80, 82 through the interior of the housing 40 and finally to the second conjugate lens 38. The conjugates 36, 38 according to this embodiment are separated by the sum of the respective focal lengths of each lens.

As noted above and according to one embodiment, the first conjugate lens 36 has an effective focal length of approximately 150 mm and the second conjugate lens 38 has an effective focal length of approximately 63 mm. Therefore, the total folded distance between the first and second conjugate lenses 36, 38 is approximately 213 mm. In another embodiment, the first conjugate lens 36 is a plano-convex element having a focal length of about 150 millimeters and the second conjugate lens 38, also a plano-convex element, has a focal length of about 88.9 millimeters, providing a total distance therebetween of about 238.9 millimeters. Other embodiments are possible. For example, it will be appreciated that the first conjugate lens 36, second conjugate lens 38, first mirror 80, and second mirror 82 may be selected and adjusted as desired within the apparatus of FIG. 8 to achieve the desired distance (e.g., 238.9 millimeters). In this example, one or more components within the apparatus of FIG. 8 may requirement adjustment and/or removal.

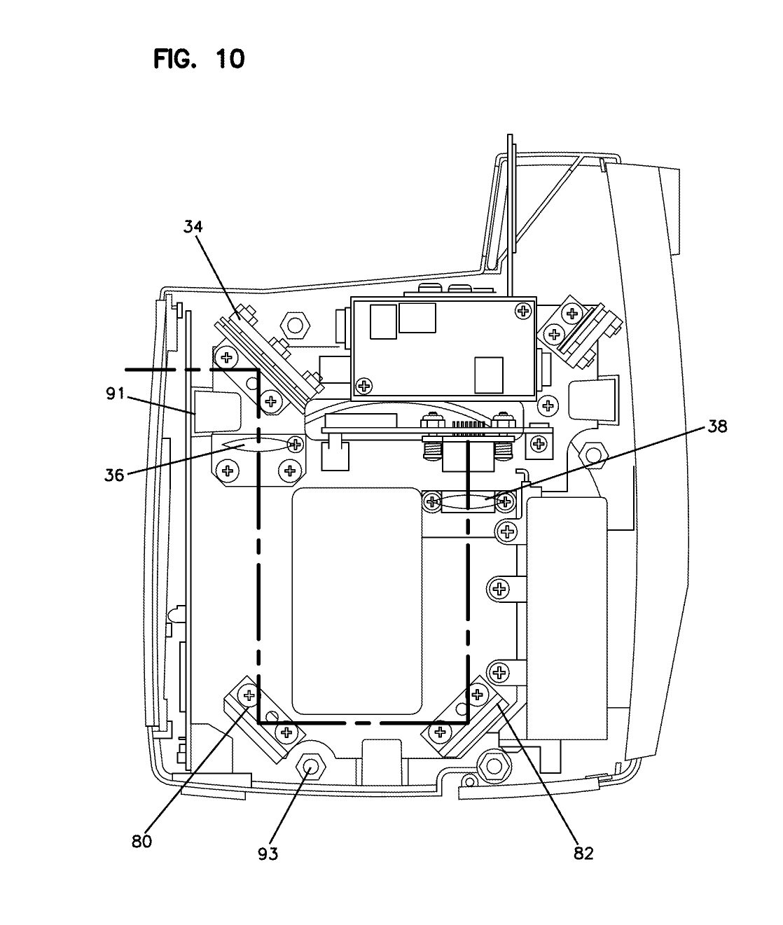

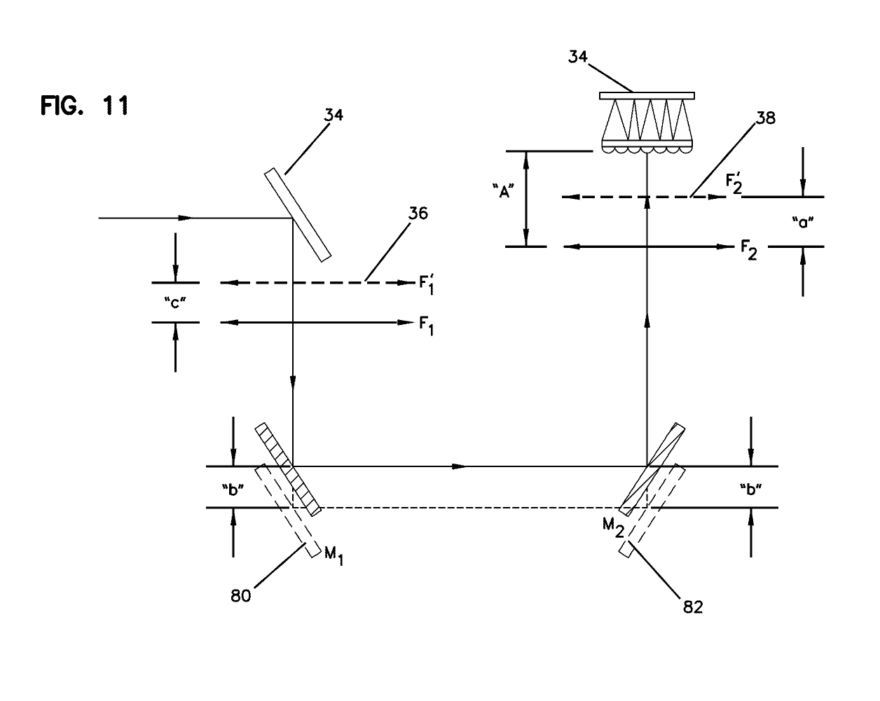

For example, referring now to FIGS. 10 and 11, the apparatus of FIG. 8, along with respective elements contained therein, are shown. More specifically, FIG. 10 shows the apparatus of FIG. 8 in a side view, and FIG. 11 shows adjustment of the first conjugate lens 36, second conjugate lens 38, first mirror 80, and second mirror 82 to achieve a desired separation distance of approximately 238.9 millimeters, from a previous separation distance of approximately 213 mm.

In this example, the first conjugate lens 36 (referred to as F1 and F1′) can be moved up approximately 6.0 mm maximum (0<c<6 mm); however, the position of damper 91 must be changed. The second conjugate lens 38 (referred to as F2 and F2′) can be moved up 11.4 mm maximum. (0<a<11.4 mm); however, to keep the “A=17 mm” unchanged, the electronic sensor 74 should be moved the same distance. The first mirror 80 (referred to as M1) can be moved down 5.0 mm maximum (0<b<5.0 mm), but position of bolt 93 should be moved to another position; otherwise the beam might be blocked. The second mirror 82 (referred to as M2) also can be moved down 5.0 mm maximum, (0<b<5.0 mm). Other adjustments might be required as well.

Referring now again to FIG. 8, to insure that the proper working distance (40 cm according to this embodiment) is established between the first conjugate lens 36 and the eye 16, an ultrasonic distance measuring device 98 is included which provides an audible signal when the instrument is located at the proper distance. Alternately, distance measurement or range finding means such as, but not limited to, time of flight, phase detection, (e.g. ultrasonic, RF, IR) triangulation, or converging projections can be used to guide the user to position the device at the proper working distance. These distances can also be captured by the electronic sensor or microprocessor (not shown) to incorporate during the calculation of refractive error to improve the accuracy of the measurement.

In addition, the apparatus also includes means for fixating the patient's gaze to ensure the patient's attention is directed to the port 81. According to one embodiment, a series of flashing LED's 90 are provided adjacent the port 81. In another embodiment, a signal generator (not shown) can emit an audible cue to direct the patient's gaze toward the port 81.

In use, the eye 16 is viewed through the viewing window 89 using the alignment pattern (not shown) for aiming the apparatus, ensuring proper alignment of the illumination assembly 42. The light is then projected by the laser diode 50, FIG. 5, through the illumination lens system as a substantially collimated beam into the eye 16, FIG. 1. The return beam is then generated as a representative wavefront 18 which is guided through the pair of conjugate lenses 36, 38, as well as the beam splitter 34, each of which is aligned with the microoptics array 20 along the return light path 70.

Since the electronic sensor 74 relies on the deviations -D- from zero positions, measured wavefront points must be matched with their zero positions. Marking the center lenslet of the microoptics array 20 (or other key location) can be done to simplify registration of the microoptics array in that only a portion of the array is actually impinged upon by the generated wavefront 18, FIG. 1. The marking can be accomplished by several different approaches, such as by removal or blackening of the center or other lenslet, or by color encoding any number of the lenslets by conventional means, such as using a filter, etc. Registration of the microoptics array 20 could also be alternately performed by flickering at least one lenslet image, using an LCD (not shown) or other known method, such as replacement of the lenslet with an LED or a test target. This would allow the image of the microoptics array 20 to be easily correlated to a calibration image.

Modifications to the above system layouts can be easily imagined for folding either the return or the illumination light path or viewing path in order to optimally size the housing 40. In addition, the instrument can be powered by batteries 94 provided in the interior of the housing 40.

Another embodiment of the present application employing the identical optical subassemblies 42, 44, 46 is herein described with reference to FIG. 9, in which similar parts are labeled with the same reference numerals for the sake of convenience. According to one embodiment, there is disposed a housing (not shown) having a support plate 103 to which the components of the present assembly are attached by conventional means. The system includes an illumination housing 79 including a contained laser diode and suitable optics to project a beam through a beam splitter 34 which directs the output of the laser diode toward the eye 16, FIG. 1, of interest. In this instance, only a single folding mirror 106 is disposed between the first and second conjugate lenses 36, 38, thereby only folding the return path once. A viewfinder portion (not shown) is attached to a mount 108 which is elevated so as to allow the viewing axis to be obliquely angled relative to the illumination axis.

The second conjugate lens 38, according to this embodiment, is attached to an adjustable block 110 and includes a spacer 112 linking each with the microoptics array and the electronic sensor, the details of each also being the same as those described with respect to FIG. 8.

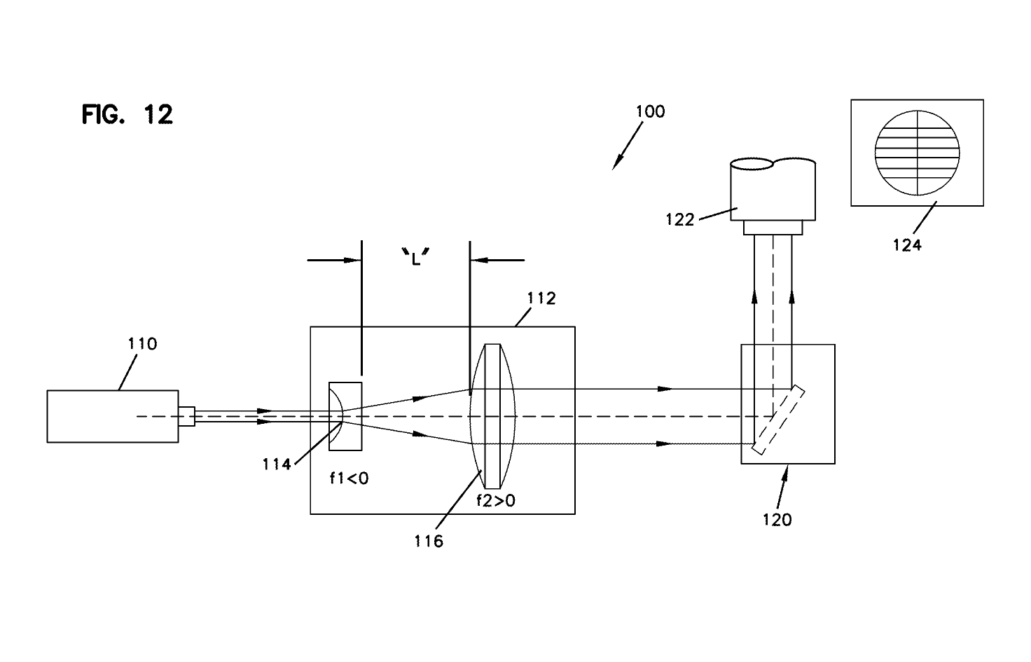

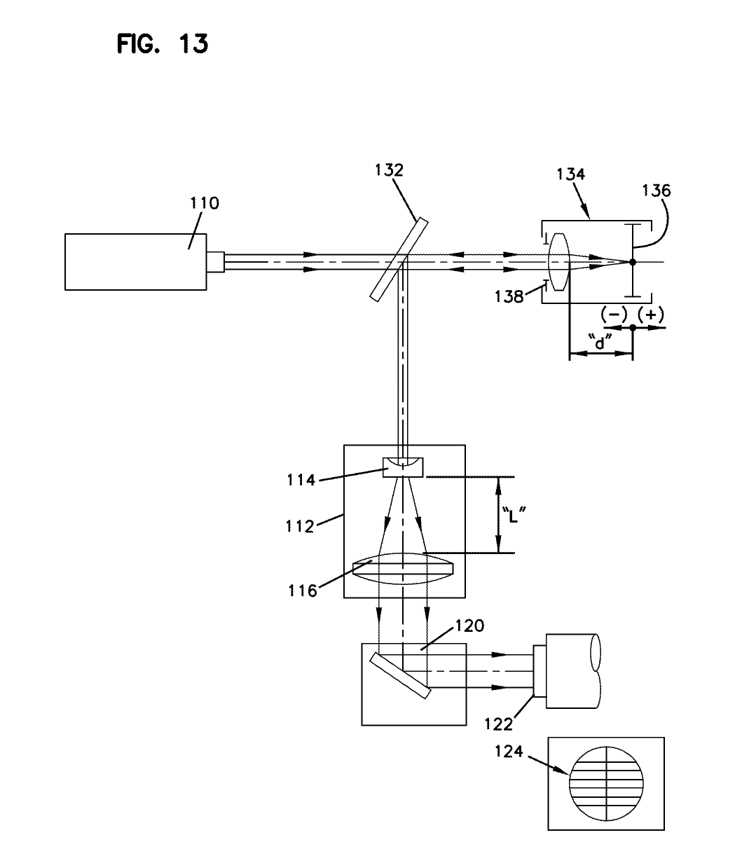

Referring now to FIGS. 12 and 13, a system 100 for calibrating the apparatus is shown. In the example, a collimated laser source 110 is used. One example of such a laser source 110 is an He—Ne laser @ 632 nm, preferably centered at a specified wavelength, such as 785 nm. Other configurations are possible.

The laser source 110 is directed to a beam expander 112 with lenses 114, 116. The output of the beam expander 112, in turn is directed to a collimation tester 120. A digital camera 122 displays the output of the collimation tester 120 on a display 124. The display shows the interference fringes formed inside the collimation tester 120.

As shown in FIG. 13, a beam splitter 132 is positioned between the laser source 110 and the beam expander 112. In this example, the beam splitter 132 is a 50/50 beam splitter, although other configurations are possible.

Also included is an adjustable face eye 134. In this example, the fake eye 134 has a 17 mm off-the-shelf lens 138 and a diffuse retinal plane (vellum) 136 that can slide back and forth along the optical axis. The fake eye 134 is adjustable to mimic aspects of a human eye so that the apparatus can be calibrated according to the method below.

The example method for calibration using the system 100 involves two steps. In step 1, the spacing “L” between the two lenses 114, 116 of the collimation tester 112 is adjusted and locked when the display 124 shows a collimated beam pattern.

In step 2, the collimation tester 112 prepared at step 1 is inserted into the setup including the beam splitter 132 and the fake eye 134. The spacing “d” between the fake eye lens 138 and the vellum 136 is adjusted and locked when the display 124 shows a collimated beam pattern. The spacing “d” corresponds to the back-focal length of the fake eye lens at the wavelength of the illumination beam.

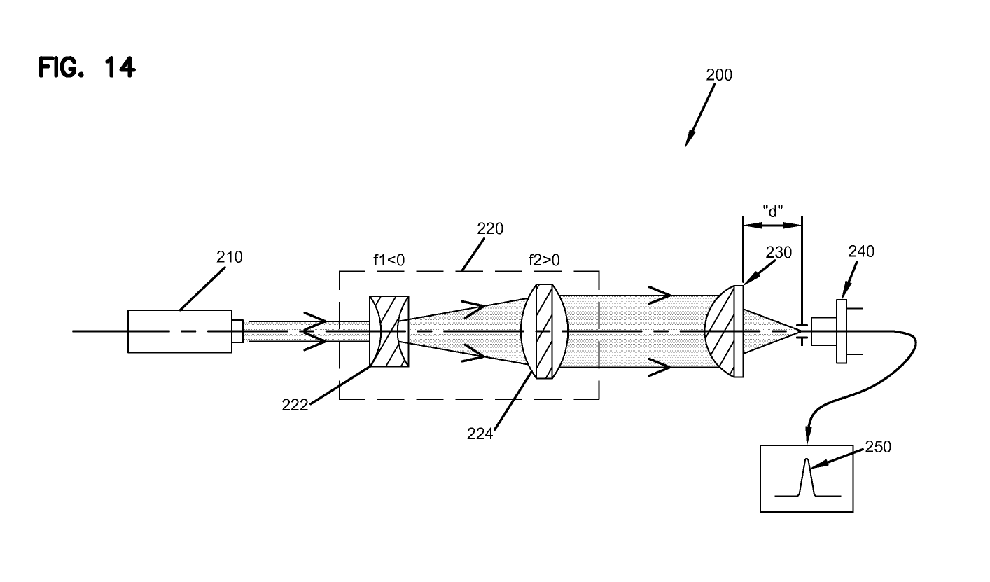

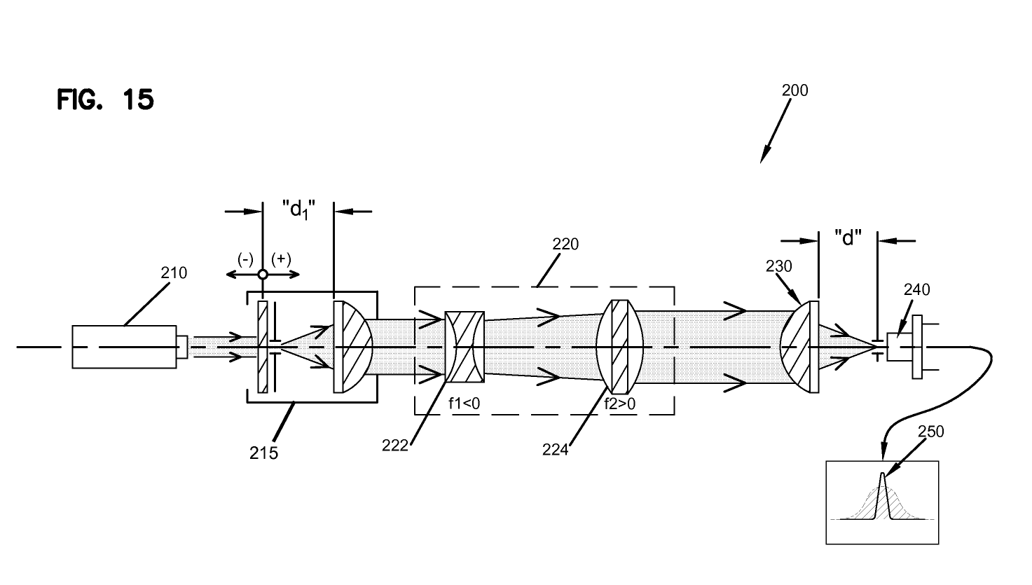

Referring now to FIGS. 14 and 15, another example system 200 for calibrating the apparatus is shown. The system 200 includes a collimated laser source 210 centered at λ=785 nm, and a beam expander 220 with lenses 222, 224. A gage lens 230 focuses the light and is positioned a distance “d” from a light meter 240 (UDT meter) with a pinhole aperture in front of it. An analog display 250 is connected to the light member 240.

There are two steps used in the calibration protocol associated with the system 200.

In step 1, the operator slides back and forth the light meter 240, changing the distance “d” until the display reports maximum intensity signal. This signal corresponds to a setting where the beam entering the gage lens 230 is perfectly collimated (i.e., “zero” diopter signal). The operator then locks in place the location of the light meter 240, which fixes the distance “d.”

In step 2, the operator inserts a fake eye assembly 215 including a diffuser and fake eye lens into the setup, and adjusts a distance “d1” therebetween until the signal intensity is maximized. This sets the “zero” diopter fake eye signal and corresponds to the nominal distance “d1.” Next, the operator varies the nominal distance “d1” and records the corresponding drop in signal intensity. The drop in signal intensity can be correlated with the departure of the fake eye 215 from the “zero” diopter condition. A lookup table can be generated enabling one to calibrate the fake eye, that is, to associate a given diopter value to the drop in signal intensity.

Other configurations and methods can be used to calibrate the apparatus.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. An apparatus for determining refractive eye aberrations, comprising:
a housing;
an illumination source positioned in the housing and configured to project a beam of light into an eye of a patient along an illumination axis, the beam forming a secondary source on a back portion of the eye for a return light path of an outgoing wavefront from the eye;
a sensor positioned in the housing and along the return light path, the sensor including a light detection surface;
a first lens and a second lens each positioned in the housing along the return light path, wherein the first lens includes a first focal length of about 150 millimeters and the second lens includes a second focal length of about 88.9 millimeters, and wherein the first and second lens are separated by a distance of about 238.9 millimeters;
an optics array positioned between the sensor and the first and second lens in the housing along the return light path, wherein the optics array includes a plurality of lenslets positioned to focus portions of the wavefront onto the light detection surface, and wherein the sensor is configured to detect deviations in positions of the focus portions impinging the light detection surface to determine aberrations of the wavefront; and
a viewer positioned in the housing and configured to align the eye with the illumination axis.

2. The apparatus of claim 1, wherein a range of measurable diopters of the eye is about −10 diopters to about +10 diopters.

3. The apparatus of claim 1, wherein the illumination source, sensor, optics array, first lens, and second lens are each fixedly coupled in the housing.

4. The apparatus of claim 1, further comprising an ultrasonic sensor positioned on the housing, the ultrasonic sensor configured to produce at least one audible signal based on a distance between the housing and the eye.

5. The apparatus of claim 1, wherein the viewer is positioned along a viewing axis, the viewing axis arranged at an oblique angle relative to the illumination axis.

6. The apparatus of claim 5, wherein the viewer includes an aiming mechanism, the aiming mechanism including an alignment pattern and a projecting mechanism.

7. The apparatus of claim 6, wherein the projecting mechanism is configured to project the alignment pattern along the viewing axis onto the back portion of the eye.

8. The apparatus of claim 1, wherein the illumination source includes a laser diode.

9. The apparatus of claim 8, wherein the laser diode is configured to emit a light beam having a wavelength in a range of about 750 nanometers to about 850 nanometers.

10. The apparatus of claim 1, wherein adjacent lenslets of the plurality of lenslets are separated by a distance of about 2 millimeters or less.

11. The apparatus of claim 1, further comprising a display configured to display data measured by the light detecting surface.

12. The apparatus of claim 1, wherein the optics array is positioned at a distance of about 17 millimeters from the second lens.

13. The apparatus of claim 1, wherein the first and second lens are each a plano-convex lens element.

14. The apparatus of claim 1, wherein the sensor is a charge coupled device.

15. The apparatus of claim 1, wherein the sensor is positioned at a distance of about 8 millimeters from the optics array.

16. The apparatus of claim 1, further comprising a beam splitter configured to redirect at least a portion of light along the return light path relative to the illumination axis.

17. The apparatus of claim 1, wherein the illumination source includes an adjustment mechanism configured to focus light onto the back of the eye.

18. The apparatus of claim 1, further comprising a fake eye configured to calibrate the apparatus.

19. A method of measuring refractive eye error, comprising:
projecting a beam of light into an eye, the light producing a secondary source and generating a wavefront from the eye along a return light path;
directing the wavefront through a first lens and a second lens onto an optics array having a series of planarly positioned lenslet elements, wherein the first lens includes a first focal length of about 150 millimeters and the second lens includes a second focal length of about 88.9 millimeters, and wherein the first and second lens are separated by a distance of about 238.9 millimeters;
focusing incremental portions of the wavefront passing through the lenslet elements onto an imaging substrate; and
measuring deviations in the incremental portions of the wavefront on the imaging substrate to measure refractive error in the eye.

20. An apparatus for determining refractive eye aberrations, comprising:
a housing;
a laser diode positioned in the housing and configured to emit a light beam into an eye of a patient along an illumination axis, the light beam having a wavelength in a range of about 750 nanometers to about 850 nanometers and forming a secondary source on a back portion of the eye for a return light path of an outgoing wavefront from the eye;
a sensor positioned in the housing and along the return light path, the sensor including a light detection surface;
a first lens and a second lens each positioned in the housing along the return light path, wherein the first lens includes a first focal length of about 150 millimeters and the second lens includes a second focal length of about 88.9 millimeters, and wherein the first and second lens are separated by a distance of about 238.9 millimeters;
an optics array positioned between the sensor and the first and second lens in the housing along the return light path, wherein the optics array includes a plurality of lenslets positioned to focus portions of the wavefront onto the light detection surface, and wherein the sensor is configured to detect deviations in positions of the focus portions impinging the light detection surface to determine aberrations of the wavefront;
an ultrasonic sensor positioned on the housing, the ultrasonic sensor configured to produce at least one audible signal based on a distance between the housing and the eye;
a viewer positioned in the housing and configured to align the eye with the illumination axis, wherein the viewer is further positioned along a viewing axis, the viewing axis arranged at an oblique angle relative to the illumination axis;
a display configured to display data measured by the light detecting surface; and
a fake eye including a lens and a vellum, wherein a space between the lens and vellum is adjustable to calibrate the apparatus;
wherein a range of measurable diopters of the eye is about −10 diopters to about +10 diopters.
